(19) United States
(12) Patent Application Publication
(10) Pub. No.: US 2006/0189962 A1
(43) Pub. Date: Aug. 24, 2006

(54) Urinary Drainage Bag for Urinary Diversion to the Bellie Button

(76) Inventor: Bonnie Beth Burtoft, Charlotte, NC (US)

Correspondence Address: THE INVENTORS NETWORK, INC., 332 ACADEMY STREET, CARNEGIE, PA, 15106, US

(21) Appl. No.: 10/906,506

(22) Filed: Feb. 23, 2005

(51) Int. Cl. A61M 27/00; A61F 5/44

(52) U.S. Cl. 604/544; 604/334

(57) ABSTRACT

A urinary drainage bag for draining urine from the belly button as a result of a urinary diversion includes a urine collection bag, a reflux chamber attached to the collection bag for accommodating urine backflow and working out bubbles, a multi-size catheter tip extending upwardly from the reflux chamber for attachment to a transfer or catheter tube, a urine discharge opening adjacent the lower end of the collection bag for draining urine from the collection bag, and a fluid measurement scale imprinted on the front of the collection bag for measuring the amount of urine held within the urine collection bag. The urinary drainage bag also includes a tether adjacent the upper end of the collection bag for conveniently hanging the collection bag, and a handle and mounting hook are also attached to the upper end of the collection bag for supporting the urinary drainage bag on a bed railing while cathing.

The present invention pertains to portable medical collection and storage bags, and more particularly pertains to a portable urine drainage bag for use by an individual that has undergone appendico or bladder augmentation surgery.

### BACKGROUND OF THE INVENTION

A number of physical conditions and surgical procedures require the use of a catheter to evacuate and empty the bladder. A commonly used type of catheter is the Foley apparatus, and it includes the various pieces of equipment for making the insertion into the body cavity, inflation of the equipment to retain it within the body cavity, the fluid transfer equipment, and the equipment for making the connection to some type of storage container such as a plastic bag. With the emphasis on taking charge of one's own health care, patients and individuals are increasingly taking on the responsibility of monitoring, implementing and maintaining their own catherization—“cathing”—routines and schedules. One type of condition that requires catherization for draining the bladder is appendico or bladder augmentation surgery. This type of surgery often is the consequence of an individual sustaining an injury that leaves him physically disabled as a paraplegic, quadriplegic or triplegic. In order to properly drain the bladder a urinary diversion must be made through the individual's belly button for accommodating the catherization equipment. This condition requires effort to maintain as the urine is often drained into a tall container or cylinder, and the physical condition of the individual makes it difficult to avoid the mess, spillage and odors that result during the catherization process, and especially when the container must be emptied and replaced. These problems are compounded for physically disabled individuals that may have only one hand available and usable for one-hand cathing.

Thus, the prior art discloses a number of medical fluid containers and assemblies for undertaking and assisting in the catherization process and in the wider field of general fluid drainage from body orifices and organs.

For example, the Eisenberg patent (U.S. Pat. No. 3,473,532) discloses a fluid container bag wherein a self-closing valve is disposed within the sidewalls of the container for making a fluid-tight seal.

The Kurtz et al. patent (U.S. Pat. No. 4,105,031) discloses an expansion chamber for a pleural drainage device that includes an expansion chamber securely attachable to the drainage device, and a connecting chamber that is attached to the expansion chamber and is connectible to a body cavity at a location different from that of the drainage device.

The Repschlager patent (U.S. Pat. No. 5,207,661) discloses a body fluid drainage assembly that includes a drip chamber for collecting CSF fluids and a drainage tube for draining such fluids. In addition, an overflow tube is connected to the drainage tube for handling overflow situations.

The Clendenning patent (U.S. Pat. No. 5,211,642) discloses a Chambers drainage system that includes at least one ten liter overflow drainage bag connected to a Foley catheter apparatus for providing a closed drainage system from the site to the overflow drainage bag.

The Magram patent (U.S. Pat. No. 5,772,607) discloses a CSF sampling apparatus wherein the container and the sampling apparatus provide a completely enclosed sampling environment for protecting against external contamination, and wherein the sampling needle includes structure that prevents fluid leakage during needle removal.

The Krueger et al. patent (U.S. Pat. No. 5,772,625) discloses an external drainage unit for fluid drainage of the lumbar region or the ventricles of the patient's brain, and includes a four-way stopcock located between the drainage burette and the drainage bag.

The Espina patent (U.S. Pat. No. 5,935,115) discloses suprapubic catheter leak collection device that includes a collection bag attached by a ring member to the ostomy site and a urinary catheter that extends within the collection bag and has a distal end for insertion into the bladder and a proximal end for subsequent removal of the urine from the catheter.

Nonetheless, despite the ingenuity of the above devices, there still remains the need for a urinary drainage system that provides physically disabled patients with an easy and convenient means of draining their bladder while minimizing or eliminating spillage and the unpleasant odors that often accompanies such activity.

### SUMMARY OF THE INVENTION

The present invention comprehends a urinary drainage bag for individuals that have undergone appendico or bladder augmentation surgery, and is especially usable for one hand cathing by physically disabled individuals such as paraplegics, quadriplegics or triplegics.

The urinary drainage bag includes a flexible plastic urine collection bag with a transparent reflux chamber mounted at its upper end. A short tube extends upwardly from the reflux chamber, and mounted to the short tube is a multi-size tip for accommodating different sizes and diameters of catheters that are interconnected to the individual's belly button for draining urine from the bladder as the result of the urinary diversion. Located at the lower portion of the urine collection bag is discharge opening for emptying the urine from the urine collection bag and imprinted on the front surface of the collection bag is a fluid measurement scale in 100 cc gradations.

A tether is secured at the upper portion of the urine collection bag for ease in carrying and transporting the urinary drainage bag to different locations and sites. In addition, a handle is also mounted at the upper portion of the urine collection bag, and projecting from the handle is a mounting hook for placing the urinary drainage bag on the railing of a bed. The individual can also hold the mounting hook while cathing with the urinary drainage bag. The urinary drainage bag also comes with a carry bag into which the urinary drainage bag can be placed for storage and privacy when the urinary drainage bag is not being used.

It is an objective of the present invention to provide a urinary drainage bag that includes measurement markings that allows the individual to easily monitor and measure urine output.

It is another objective of the present invention to provide a urinary drainage bag that is capable of storing up to 1,000 cc's of urine resulting in less maintenance and bother for the individual.

It is still another objective of the present invention to provide a urinary drainage bag that allows the individual to quickly and easily attach the urinary drainage bag to catheters of various sizes and diameters.

It is still yet another objective of the present invention to provide a urinary drainage bag that can last approximately two months before the bag needs emptied and replaced.

It is still yet a further objective of the present invention to provide a urinary drainage bag that diminishes and reduces the likelihood of unpleasant odors escaping from the bag.

A further objective of the present invention is to provide a urinary drainage bag that can be easily and quickly emptied of the contents held therein.

A still further objective of the present invention is to provide a urinary drainage bag that is easily portable so that the individual can use the drainage bag away from home, such as on trips and vacations.

Yet a further objective of the present invention is to provide a urinary drainage bag that includes a structural element or feature for accommodating any backflow of urine.

Yet still a further objective of the present invention is to provide a urinary drainage bag that prevents the mess and smell that often accompanies such drainage and is especially suitable for one-hand cathing by tri-plegics.

Yet another objective of the present invention is to provide a urinary drainage bag that includes structural elements that allow a transparent view of urine flow into the bag and provide the individual with the ability to work out bubbles that may accompany the urine flow.

These and other objects, features and advantages will become apparent to one skilled in the art upon a perusal of the following detailed description read in conjunction with the appended drawing figures.

### BRIEF DESCRIPTION OF THE DRAWINGS

FIG. 1 is a front elevational view of the urinary drainage bag of the present invention illustrating the attachment of the bag to a catheter that drains from the belly button of the individual;

FIG. 2 is a perspective view of the urinary drainage bag of the present invention illustrating the attachment of the catheter to the tube projecting from the upper portion of the drainage bag;

FIG. 3 is a perspective view of the urinary drainage bag of the present invention illustrating the placement of the urinary drainage bag on the end railing of a bed; and

FIG. 4 is a perspective view of the urinary drainage bag of the present invention illustrating the use of a lower discharge tube tied off by a metal clamp for draining urine from the urinary drainage bag.

### DETAILED DESCRIPTION OF THE PREFERRED EMBODIMENT

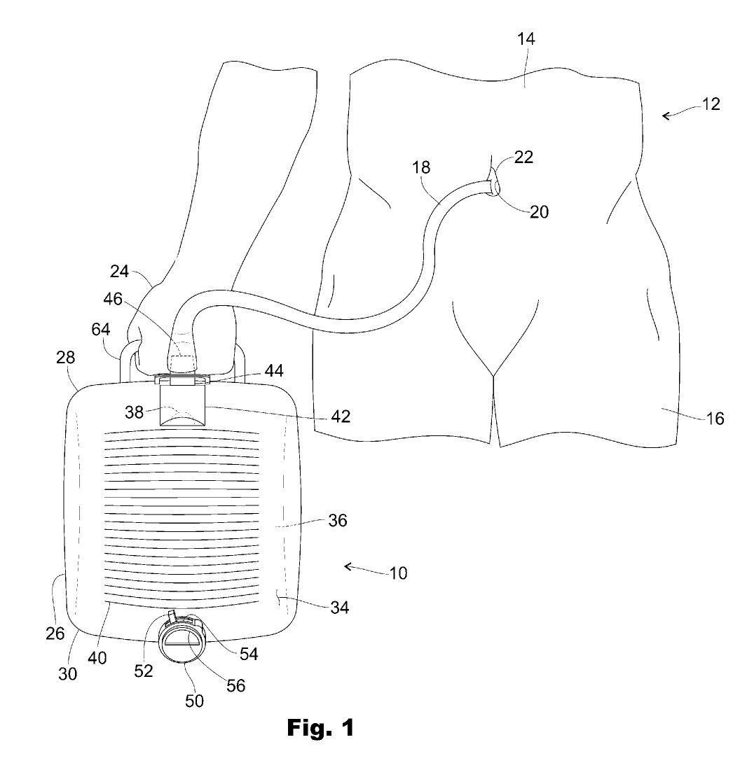

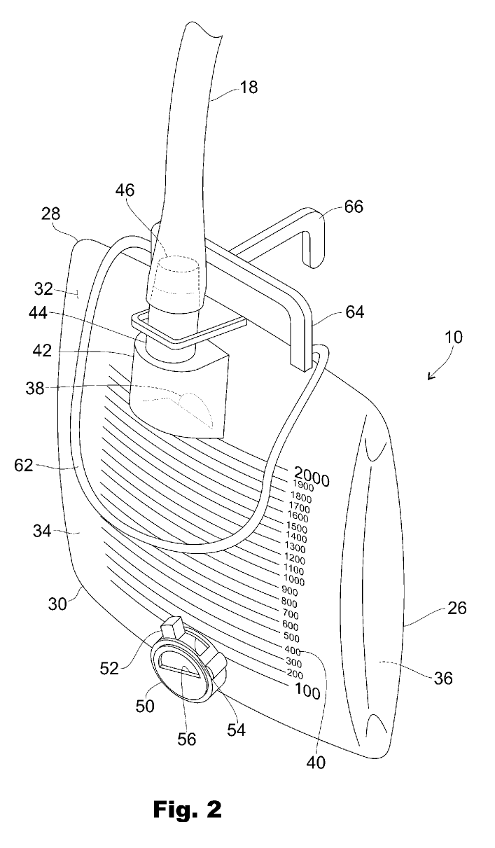

Illustrated in FIGS. 1-4 is a urinary drainage bag 10 for the urinary diversion to the individual's belly button for use after appendico or bladder augmentation surgery. The urinary drainage bag 10 provides physically disabled individuals, such as paraplegics, quadriplegics, and triplegics, with an easy and convenient means for emptying their bladder while avoiding spillage or the escape of noxious odors. The present invention also provides triplegics with the ability to undertake one-hand cathing, and is used for attachment to a catheter for draining urine by the urinary diversion.

Thus, shown in FIGS. 1-4 are the individual's lower torso 12, abdomen 14 and upper thighs 16 of both legs. A transfer or catheter tube 18 is inserted through a slot 20 formed at the belly button 22 for interconnection to the bladder, with the distal end of the catheter 18 attached to the urinary drainage bag 10 for draining of the urine. The individual is also shown as holding the urinary drainage bag 10 by the hand 24. The urinary drainage bag 10 includes a flexible, plastic urine collection bag 26 for containing and holding therein the individual's urine. The urine collection bag 26 includes an upper end 28, an opposite lower end 30, and a rear panel 32 joined to a front panel 34 at their mutual peripheral edges to form an interior urine-holding chamber 36. The front panel 34 includes a urine aperture 38 for allowing the urine to flow into the urine-holding chamber 36. In addition, imprinted on the front panel 34 is a fluid measurement scale 40 that can be marked off in various ways; and in the representative measurement scale 40 the gradations are in units of 100 cubic centimeters and the urine collection bag 26 is capable of holding at least 1,000 cubic centimeters of urine before it requires emptying. The easily discernible measurement scale 40 allows the individual to keep continuous track of the amount of urine held within the urine collection bag 26 at any given time, and the ability of the urine collection bag 26 to hold at least 1,000 cubic centimeters of urine results in less mess and maintenance for the individual.

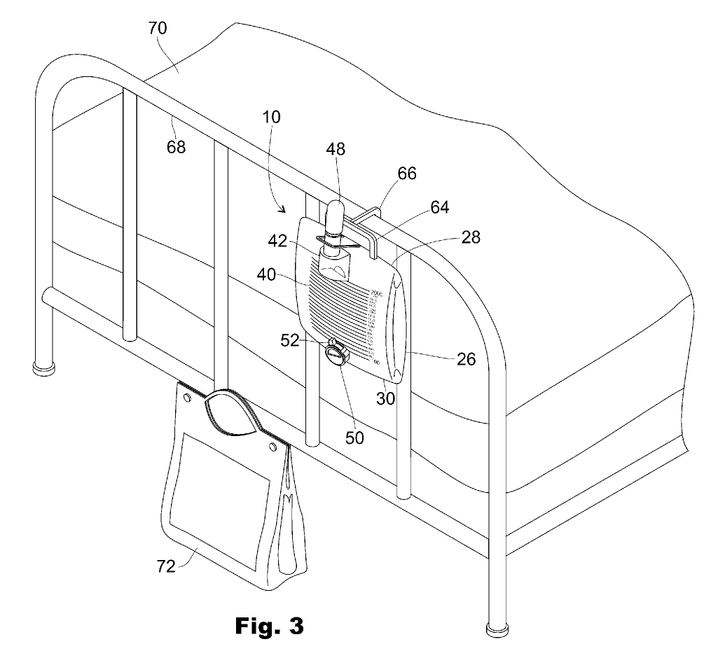

As shown in FIGS. 1-4, attached to the front panel 34 adjacent the upper end 28 thereof is a transparent reflux chamber 42. The reflux chamber 42 registers with the urine aperture 38 to allow urine to flow from the catheter 18 through the reflux chamber 42 and through the urine aperture 38 for containment within the urine-holding chamber 36. The reflux chamber 42 is raised from or projects outwardly from the front panel 34 so that the flow of urine therethrough is easily discernible by the individual. The reflux chamber 42 also provides several other significant functions for the urinary drainage bag 10 in so far as the reflux chamber 42 accommodates any urine backflow, facilitates the working out and dissipation of any air bubbles that may be entrained with the urine flow, and diminishes the likelihood of offensive odors escaping from the urine collection bag 26. Mounted to the reflux chamber 42 is a short or intermediate tube 44, and mounted to the intermediate tube 44 is a multi-size tip 46 to which the end of the catheter 18 is secured; the multi-size tip 46 allows for the attachment thereto of catheters 18 having various sizes, diameters and gauges. A cap 48 is included for placement on the multi-size tip 46, as shown in FIG. 3, in order to maintain the cleanliness of the tip 46 during periods of non-use of the urinary drainage bag 10.

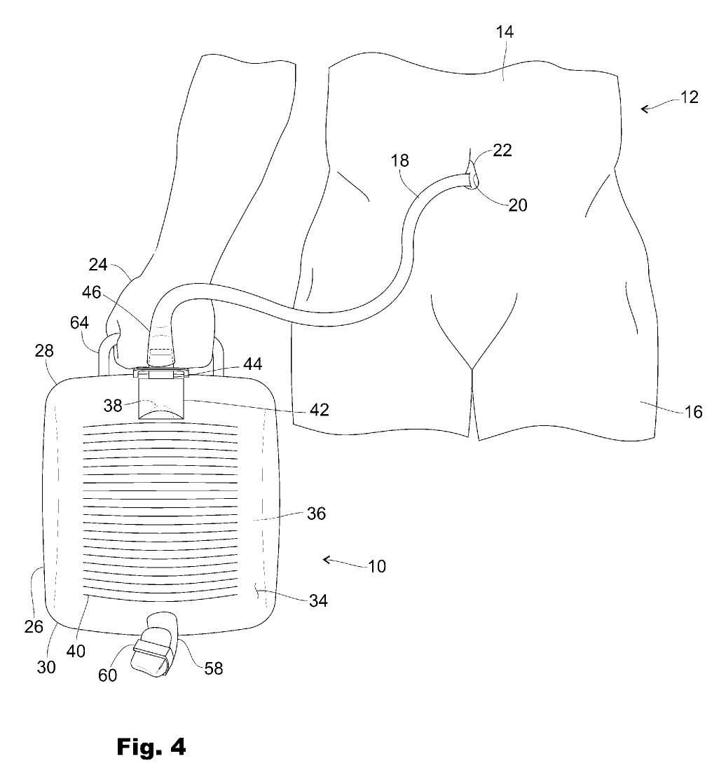

The urinary drainage bag 10 of the present invention includes a urine discharge means to drain or empty urine from the collection bag 26 when desired by the individual; although it should be noted that the urine collection bag 26 could last up to two months before being replaced. FIGS. 1-3 illustrate a first preferred embodiment of the urine discharge means and FIG. 4 illustrates a second preferred embodiment of the urine discharge means.

The urine discharge means of FIGS. 1-3 includes a drain cap 50 mounted to the front panel 34 at the lower end 30 of the front panel 34 for sealably covering the urine discharge opening. A movable latch 52 is mounted within a slot 54 on the drain cap 50 and the latch 52 is selectively movable between an open and a close position for opening and closing a baffle or shutter 56 that registers with the discharge opening. Thus, urine can be emptied from the urine collection bag 26 by moving the latch 52 to the open position thereby allowing urine to flow through the discharge opening and eliminating any spillage or mess as well as avoiding any contact of the discharging urine with the individual's hands or fingers.

FIG. 4 illustrates a flexible tube 58 mounted to the lower end 30 of the urine collection bag 26 and in registration with the discharge opening for allowing urine to flow through the flexible tube 58 for emptying the urine collection bag 26. A clamp member 60, such as a metal clamp, is slidably inserted on the flexible tube 58 to close off the discharge opening when the urine collection bag 26 is being used for cathing. When the individual desires to empty the urine collection bag 26, the clamp member 60 is simply removed from the flexible tube 58 thus permitting the urine to empty therefrom.

As shown in FIGS. 1-4, the urinary drainage bag 10 includes a flexible cord or tether 62 that allows the individual to easily transport the urinary drainage bag 10 or to hang the urinary drainage bag 10 from a convenient location. In addition, the urinary drainage bag 10 includes a handle 64 mounted to the upper end 28 of the urine collection bag 26. Pivotally connected to the handle 64 is a mounting hook 66 for placement or hooking on to furniture, such as the railing 68 of a bed 70, so that the urinary drainage bag 10 can be supported thereon during cathing. The handle 64 provides the individual with another convenient method of carrying and transporting the urinary drainage bag 10, and the mounting hook 66 can be folded alongside the handle 64 to facilitate the transport or storage of the urinary drainage bag 10. Finally, a carry bag 72 is included in which the urinary drainage bag 10 can be stored so that the individual can obtain privacy in the use of the urinary drainage bag 10 when the individual is on vacation or if the individual utilizes the urinary drainage bag 10 during periods of hospitalization.

Although a specific embodiment of the invention has been shown and described, numerous alterations, variations, and modifications will be apparent to those skilled in the art without departing from the spirit of the invention or the scope of the intended claims.

What is claimed is:

1. A urinary drainage bag connected to a catheter for draining urine from a urinary diversion to the belly button, comprising:
a urine collection bag having an upper end, an opposite lower end, and a front panel;
a reflux chamber mounted adjacent the upper end of the urine collection bag for dissipating air bubbles that may accompany the flow of urine and for accommodating any urine backflow;
a multi-size tip extending upwardly from the reflux chamber for attachment to the catheter so that urine can flow through the catheter and reflux chamber and into the urine collection bag;
a urine discharge means located at the lower end of the urine collection bag for emptying urine from the urine collection bag after a given amount of urine has collected within the urine collection bag;
a tether attached to the upper end of the urine collection bag for hanging the urine collection bag from a convenient location;
a handle mounted to the upper end of the urine collection bag;
a mounting hook attached to the handle and extending rearward therefrom so that the mounting hook can be hooked onto the railing of a bed while the individual is using the urinary drainage bag for cathing; and
a fluid measurement scale imprinted on the front panel of the urine collection bag providing the individual with an accurate measurement of the amount of urine held within the urine collection bag.

2. The urinary drainage bag of claim 1 further comprising a cap for closing off the multi-size catheter when the urine collection bag is not in use.

3. The urinary drainage bag of claim 2 wherein the reflux chamber is transparent and projects outwardly from the front panel of the urine collection bag to facilitate the flow of urine into the urine collection bag and to allow the individual to view the urine flow.

4. The urinary drainage bag of claim 3 wherein the mounting hook is pivotally secured to the handle so that the mounting hook can be folded alongside the handle when the urine collection bag is not in use.

5. The urinary drainage bag of claim 4 wherein the urine discharge means includes a discharge tube attached to the urine collection bag adjacent the lower end for allowing the emptying of urine from the urine collection bag.

6. The urinary drainage bag of claim 5 wherein the urine discharge means includes a clamp member removably insertable to the discharge tube so that insertion of the clamp member closes the discharge tube and removal of the clamp member opens the discharge tube for emptying urine from the urine collection bag.

7. The urinary drainage bag of claim 6 wherein the urine discharge means includes a drain cap mounted to the front panel at the lower end of the urine collection bag.

8. The urinary drainage bag of claim 7 wherein the urine discharge means includes a latch mounted to the drain cap and movable between an open position and a closed position for opening the drain cap to empty urine from the urine collection bag and for closing the drain cap so that urine can be held within the urine collection bag.

9. A urinary drainage bag for connection to a catheter so that urine can be drained from a urinary diversion to the belly button, comprising:
a flexible, plastic urine collection bag having an upper end, an opposite lower end, and a front panel;
a reflux chamber mounted at the upper end of the urine collection bag for dissipating air bubbles that accompany urine flow and for accommodating any urine backflow;
a multi-size tip extending upwardly from the reflux chamber for attachment to the catheter so that urine can flow through the catheter and through the reflux chamber into the urine collection bag;
a urine discharge means located at the lower end of the urine collection bag for emptying urine from the urine collection bag after a given amount of urine has collected within the urine collection bag;
a handle mounted to the upper end of the urine collection bag;
a mounting hook attached to the handle and extending rearward therefrom so that the mounting hook can be placed onto the railing of a bed for supporting the urinary drainage bag thereon as the individual is using the urinary drainage bag for cathing; and
a fluid measurement scale imprinted on the front panel of the urine collection bag for providing the individual with an accurate measurement of the amount of urine held within the urine collection bag.

10. The urinary drainage bag of claim 9 further comprising a tether attached to the upper end of the urine collection bag for suspending the urine collection bag from any convenient location.

11. The urinary drainage bag of claim 10 further comprising a cap for closing off the multi-size catheter when the urine collection bag is not in use.

12. The urinary drainage bag of claim 11 wherein the reflux chamber is transparent and projects outwardly from the front panel of the urine collection bag to facilitate the flow of urine into the urine collection bag and to allow the individual to view the urine flow.

13. The urinary drainage bag of claim 12 wherein the mounting hook is pivotally secured to the handle so that the mounting hook can be folded alongside the handle when the urine collection bag is not in use.

14. The urinary drainage bag of claim 13 wherein the urine discharge means includes a discharge tube extending from the lower end of the urine collection bag for allowing the emptying of urine from the urine collection bag.

15. The urinary drainage bag of claim 14 wherein the urine discharge includes a clamp member that is removably insertable on the discharge tube so that insertion of the clamp member closes the discharge tube and removal of the clamp member opens the discharge tube for draining urine from the urine collection bag.

16. The urinary drainage bag of claim 15 wherein the discharge means includes a drain cap mounted to the front panel at the lower end of the urine collection bag.

17. The urinary drainage bag of claim 16 wherein the urine discharge means includes a latch mounted to the drain cap and movable between an open position and a closed position for opening the drain cap to drain urine from the urine collection bag and for closing the drain cap so that urine can be held within the urine collection bag.
